(19) United States
(12) Plant Patent Application Publication
(10) Pub. No.: US 2017/0303455 P1
(43) Pub. Date: Oct. 19, 2017

(54) Dianthus plant named 'WP15 PIE45'

(50) ;

(71) Applicant: Plant Genetics International Ltd., Hambrook (GB)

(72) Inventor: Carolyn Grace Bourne, Devon (GB)

(73) Assignee: Plant Genetics International Ltd., Hambrook (GB)

(21) Appl. No.: 15/731,061

(22) Filed: Apr. 12, 2017

(30) Foreign Application Priority Data
Apr. 15, 2016 (QZ) 2016/0942

(51) Int. Cl. A01H 5/02

(52) U.S. Cl. CPC A01H 5/0233
USPC PLT/272

(57) ABSTRACT

A new cultivar of Dianthus plant named ‘WP15 PIE45’ that is characterized by large single flowers with an apricot-khaki colored eye delimited by a bright red-orange line, deep fimbriated petal edges, medium sweet fragrance, blooming from May through October, a compact mounding habit, glaucous blue green foliage and that is suitable for use as a flowering plant in pots and containers as well as for use as a cut flower and in bouquets, is disclosed.

Genus and species: Dianthus allwoodii hort.

Variety denomination: ‘WP15 PIE45’

### CROSS REFERENCE TO RELATED APPLICATIONS

This application claims the benefit of priority under 35 U.S.C. 119 (f) to the application for European Community Plant Variety Rights which was filed for the instant plant variety on Apr. 15, 2016, File Number 2016/0942.

### BACKGROUND OF THE NEW PLANT

The present invention relates to a new and distinct cultivar of hardy Dianthus, commonly known as Garden Pink, which is grown for use as a flowering plant for pots and containers and for planting in the gaiden and landscape. The new cultivar is known botanically as Dianthus allwoodii hort. and will be referred to hereinafter by the cultivar name ‘WP15 PIE45’.

‘WP15 PIE45’ was selected from the open pollination of a group of unnamed and unreleased seedlings of Dianthus in the group Dianthus allwoodii hort. which is characterized by large single flowers. ‘WP15 PIE45’ is the product of a long-term breeding program conducted in a cultivated area of Houndspool, Dawlish, Devon, United Kingdom. ‘WP15 PIE45’ was selected in 2012 for its large single flowers with an apricot—khaki colored eye delimited by a bright red-orange line, deep fimbriated petal edges, medium sweet fragrance and glaucous blue-green foliage.

Asexual reproduction of ‘WP15 PIE45’ was first accomplished in 2012 in a cultivated area of Houndspool, United Kingdom using the propagation method of shoot cuttings. ‘WP15 PIE45’ has been determined to be stable and reproduces true to type in successive generations of asexual propagation.

### SUMMARY

The following traits have been repeatedly observed and represent the characteristics of the new Dianthus cultivar ‘WP15 PIE45’. ‘WP15 PIE45’ has not been tested under all possible conditions and phenotypic differences may be observed with variations in environmental, climatic and cultural conditions, however, without any variance in genotype.
1. ‘WP15 PIE45’ exhibits a compact mounding habit.
2. ‘WP15 PIE45’ bears single light pink-peach flowers with an apricot-khaki eye.
3. The petals of ‘WP15 PIE45’ are flat.
4. ‘WP15 PIE45’ blooms from May through October.
5. The flowers of ‘WP15 PIE45’ exhibit a medium sweet fragrance.
6. ‘WP15 PIE45’ exhibits glaucous blue green foliage.
7. At maturity, the height of the foliage mound of ‘WP15 PIE45’ is 40 cm, and the width is 30 cm to 35 cm.
8. ‘WP15 PIE45’ is perennial and is hardy at least to minus 15° Centigrade.
9. ‘WP15 PIE45’ is suitable for use as a flowering plant in pots and containers, and for use as a cut flower and in bouquets.

### BRIEF DESCRIPTION OF THE PHOTOGRAPHS

The accompanying color photographs illustrate the overall appearance of the new Dianthus variety ‘WP15 PIE45’ showing colors as true as it is reasonably possible to obtain in colored reproductions of this type. Colors in the photographs may differ from the color values cited in the detailed botanical description, which accurately describe the actual colors of the new variety ‘WP15 PIE45’.

FIG. 1 depicts an entire flowering plant of ‘WP15 PIE45’.

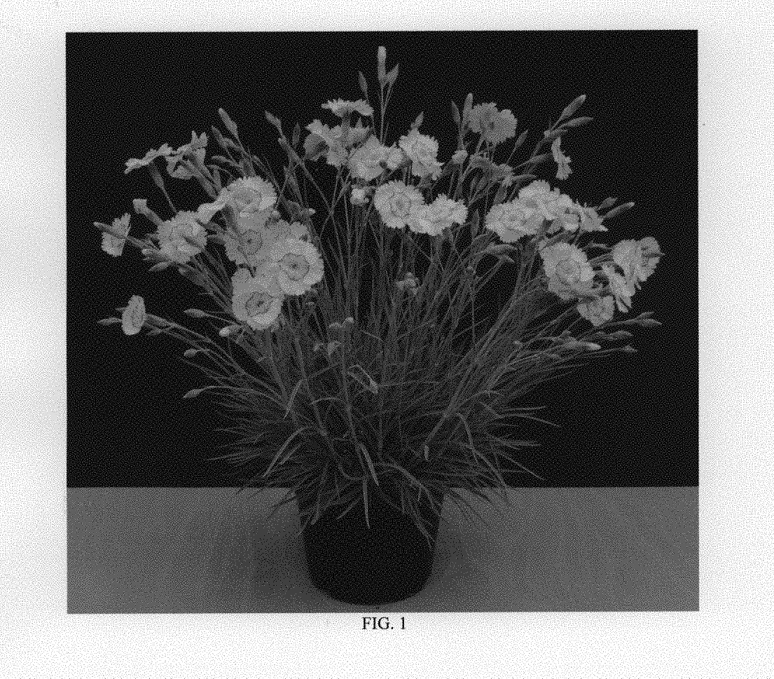

FIG. 2 depicts a close-up view of the flower of ‘WP15 PIE45’.

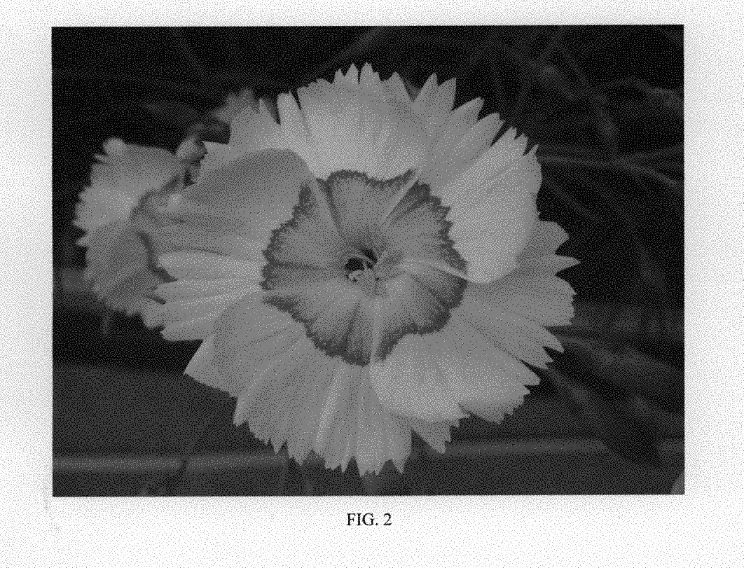

Photographs were taken from a 9 months old plant which was growing in a glasshouse in a 3-liter container at the Inventor's nursery in Dawlish, Devon, United Kingdom. No chemicals were used to treat the plants. All photographs were made using conventional techniques and although colors may appear different from actual colors due to light reflectance they are as accurate as possible by conventional photography.

### BOTANICAL DESCRIPTION OF THE PLANT

The following is a detailed description of the new cultivar ‘WP15 PIE45’. Data was collected from plants grown in 3-liter containers under an unheated glasshouse in Houndspool, Dawlish, United Kingdom. The color determinations are in accordance with the sixth edition (2015) of The Royal Horticultural Society Colour Chart of The Royal Horticultural Society, London, England, except where general color terms of ordinary dictionary significance are used. No chemicals were used to treat the plants. Growing conditions are typical to other Dianthus.
Botanical classification: Dianthus
   Variety.—‘WP15 PIE45’.
   Species.—×allwoodii hort.
   Common names.—Hybrid dianthus, Garden Pink.
   Commercial classification.—Hardy perennial.
   Use.—Ornamental plant for pots and containers.
Parentage: ‘WP15 PIE45’ resulted and was selected from the open pollination of a group of unnamed and unreleased seedlings of Dianthus in the group Dianthus allwoodii hort.
Plant description:
   Bloom period.—May to October.
   Plant habit.—Compact mounding habit.
   Plant height.—40 cm in overall height, including flower canopy.
   Plant width.—30 cm to 35 cm in width.
   Plant hardiness.—Hardy to minus 15° Centigrade.
   Type.—Perennial.
   Root system.—Fibrous.
   Propagation.—Propagation is accomplished using shoot cuttings.
   Cultural requirements.—Plant in full sun, well-drained and moderately fertile soil and keep well fertilized and watered.
   Time required to produce a rooted cutting.—5 weeks are needed to produce a rooted cutting.
   Temperature recommended for cuttings to produce roots.—The air temperature needed is a minimum of 15° Centigrade air temperature and base heat of 21° Centigrade.
   Crop time.—6 to 9 months is needed to produce a finished 3-litre container size from a well-rooted cutting.
Stem:
   Shape.—Cylindrical, solid.
   Dimensions.—10 cm in length, 4 mm diameter.
   Surface.—Glabrous and glaucous.
   Color.—145B and 145C.
   Branching.—Numerous shoots from the axils of the lower leaves.
   Internode length.—4 mm to 6 mm between nodes (before extension of peduncle).
   Node dimensions.—4 mm in diameter and 5 mm in length.
Leaf:
   Type.—Evergreen.
   Dimensions.—9 cm in length, 3 mm in width.
   Color (adaxial and abaxial surfaces).—N189B and N189C.
   Shape.—Linear.
   Division.—Simple.
   Apex.—Acute.
   Base.—Dccurrent.
   Venation.—Absent on the adaxial surface and only one central vein visible on the abaxial surface
   Margins.—Entire.
   Attachment.—Sheathing.
   Arrangement.—Opposite and spiraling up stem.
   Surfaces (adaxial and abaxial).—Glaucous.
   Fragrance.—Absent.
Flowers:
   Inflorescence.—Simple cyme.
   Type.—Single, polypetalous.
   Number.—5 to 9 per stem.
   Dimensions (including calyx).—35 mm in length and 55 mm in diameter.
   Shape.—Circular.
   Lastingness.—10 days at 20° Celsius on the plant; 8 to 10 days off the plant.
   Fragrance.—Medium, sweet.
   Flowering period.—May to October.
Peduncle:
   Shape.—Cylindrical, solid.
   Dimensions.—25 cm to 30 cm in length, 2 mm in diameter.
   Surface.—Glabrous, waxy.
   Strength.—Strong, rigid.
   Color.—N189B to N189C.
Bud:
   Shape.—Cylindrical.
   Color.—N138A to N138B on apical half and 144B to 144C on basal half (below the bracts).
   Anthocyanin.—Absent.
   Dimensions.—28 mm in length and 8 mm in width.
Petals:
   Corolla.—Upper and lower part of corolla is flat.
   Petal arrangement.—Persistent, apopetalous and overlapping.
   Margin.—Irregularly serrate.
   Indentations.—Up to 8 mm deep.
   Texture.—Soft.
   Type.—Single.
   Number of petals.—5.
   Dimensions.—56 mm in length, 35 mm in width.
   Petal shape.—Fan shape.
   Petal surfaces.—Glabrous, abaxial surface irregularly papillate.
   Petal apex.—Fan shaped, irregularly serrate margin.
   Petal base.—Strap shaped, tapering towards base.
   Color pattern.—Flower is opening into a light pink-peach main color with an apricot-khaki band delimited by a bright red-orange line with slight inward bleeding. The main color of the blade ages to almost white.
   Petal color (adaxial surface).—Ground color of blade when just opened: 69C, 69D, 49D and 36D are all present. Ground color of blade when mature: N1 55B. Color of band: 23D, 24D and 25D are all present. Color of line delimiting color of blade and band: 42A to 43A. Color of middle of strap: 145C. Color of base of strap: 145D.
   Petal color (abaxial surface).—Ground color of blade: 69C. Color of middle of strap: 145C. Color of base of strap: 145D.
Calyx:
   Dimensions.—30 mm in length and 9 mm in width.
   Shape.—Cylindrical.
   Sepals.—5, fused to 7 mm below the apex.
   Sepal dimensions.—31 mm in length and 7 mm in width.
   Apex.—Acute.
   Base.—Truncate.
   Color of sepals, outer surface.—137B fading to 137D at base.
   Color of sepals, inner surface.—139C to 139D.
   Anthocyanin.—Absent.
   Splitting.—Not observed.
Epicalyx:
   Number of Bracts.—1 pair of 2.
   Bracts shape.—Medium acuminate.
   Bracts dimensions.—12 mm in length and 6 mm in width.
   Bracts color.—N138C, N138D and 138D are all present.
   Anthocyanin.—Absent.
   Number of Bracteoles.—1 pair of 2.
   Bracteoles shape.—Medium acuminate.
   Bracteoles dimensions.—11 mm in length and 11 mm in width.
   Bracteoles color.—N138C, N138D and 138D are all present.
   Bracteoles anthocyanin.—Absent.
Reproductive organs:
   Stamen number.—10 stamens.
   Stamen dimensions.—30 mm length and 0.5 mm diameter.
   Color of filaments.—NN155D.
   Anther number.—10.
   Anther dimensions.—4 mm length and 1 mm in diameter.
   Color of anthers.—NN155D.
   Anther attachment.—Dorsifixed.
   Pollen.—Present, dust color close to 156D.
   Style number.—2.
   Style shoulder.—Absent.
   Color of style.—NN155D.
   Style dimensions.—30 mm to 35 mm in length and 1 mm in width.
   Stigma number.—One on each style.
   Stigma shape.—Linear, along the top of the style when mature, with a bilobed extremity.
   Stigma color.—Pink hue.
   Stigma dimensions.—Approximately 6 mm in length, along the style.
   Ovary position.—Superior.
   Ovary dimensions.—11 mm in length and 5 mm in diameter.
   Ovary shape.—Obovate.
   Ovary surface and texture.—Smooth and slightly ribbed.
   Ovary color.—144C and 145A at the uppermost surface, becoming paler 145C to 145D at the lowest surface.
Seed:
   Color.—202A.
   Length.—3 mm.
   Diameter.—2 mm.
   Shape.—Oval and flattened.
   Surface.—Crinkled.
   Number.—Varies, up to 20 per pod.
Diseases and pests: Susceptible to known Dianthus pests and disease but no other susceptibilities to pests or disease are known.
Comparison with parental line and known variety: The comparison with the parents is not possible as “WP15 PIE45” was selected from the open pollination of a group of unnamed and unreleased seedlings of Dianthus in the group Dianthus allwoodii hort., all characterized by large single flowers. The variety of Dianthus considered to most closely resemble ‘WP15 PIE45’ is the inventor's variety Dianthus Plant Named ‘WP15 PIE41’. In comparison with ‘WP15 PIE45’, ‘WP15 PIE41’ has slightly shorter flower stems and bears white single flowers with a lime-colored eye.

I claim:

1. A new and distinct cultivar of Dianthus plant named ‘WP15 PIE45’ as described and illustrated herein.
